(19) United States
(12) Patent Application Publication
(10) Pub. No.: US 2021/0290103 A1
(43) Pub. Date: Sep. 23, 2021

(54) USER DEVICE FOR REGISTERING DISEASE RELATED STATES OF A USER

(71) Applicant: MEDICAL DEVICE DEVELOPMENT GROUP B.V., Zevenaar (NL)

(72) Inventors: Johannes Cornelis Muller, Doesburg (NL); Peter Cornelis Godfried Nijssen, Gilze (NL)

(21) Appl. No.: 17/260,163

(22) PCT Filed: Jun. 28, 2019
(86) PCT No.: PCT/NL2019/050402

§ 371 Date: Jan. 13, 2021

(30) Foreign Application Priority Data
Jul. 16, 2018 (NL) 2021316

(51) Int. Cl. A61B 5/11; G16H 50/30; A61B 5/00

(52) U.S. Cl. CPC A61B 5/1101; G16H 50/30; A61B 5/681; A61B 5/748; A61B 5/4082; A61B 5/7282; A61B 5/0022; A61B 5/746; A61B 2562/0219; A61B 5/742

(57) ABSTRACT

A wearable electronic user device comprising a processor, a touch-sensitive display and a timer is disclosed. The device can be used to register disease-related states of the user. The processor is configured to generate a scoring scale comprising at least three distinct scores concerning disease-related states of the user. The touch-sensitive display is configured to display the scoring scale as generated by the processor enabling a user interaction with the scoring scale. The timer is configured for generating timing information for registering timing instances associated with the detected user interaction. The processor is configured for detecting the user interaction with the touch-sensitive display on or nearby the scoring scale to assign scoring information to the detected user interaction by associating the user interaction with one of the distinct scores.

### CROSS-REFERENCE TO RELATED APPLICATION

This Application is a Section 371 National Stage Application of International Application No. PCT/NL2019/050402, filed Jun. 28, 2019 and published as WO 2020/017953 A1 on Jan. 23, 2020, in English, the contents of which are hereby incorporated by reference in their entirety.

### FIELD OF THE INVENTION

The invention relates to a user device for registering disease related states of a user. The invention further relates to an external computer system for use with the user device and to a method for registering disease related states of a user aided by the user device.

### BACKGROUND OF THE INVENTION

Gradually increasing stiffness, slowness, balance problems and annoying trembling (tremor) are the most well-known complaints in Parkinson's disease. The disease is common: in people older than 55 years, 1 in 100. The cause is unknown, but lack of dopamine in the brain plays a big role. Unfortunately, the disease cannot be cured, but medicines (like levodopa or dopamine agonists) often work well for years. The dose is adjusted according to the progression of complaints. After some years of medication use, response-fluctuations occur: the nature of the disease can suddenly change several times a day. Stiffness and slowness and other typical Parkinson symptoms (off-states) occur while blood levels are too low; alternating with ‘good’ on-states at a correct level; or involuntary movements (dyskinesias) due to excessive blood levels where patients are overly active. Detailed data of the time-course of these three states, are required to allow a doctor to adjust the medication scheme, and for monitoring the effectiveness of a treatment strategy.

Measuring systems are known which are suitable for analysis of tremors of patients and which are wearable on the body of the person. However, a need exists in the art for a more convenient manner for registration of the perceived state of a patient.

### SUMMARY OF THE INVENTION

It is an aim of the invention to provide a user convenient device and method for registration of the state of the user and additional information.

To that end, one aspect of the invention pertains to a wearable electronic user device comprising a processor, a touch-sensitive display and a timer. The wearable electronic user device may e.g. be a smart phone or a smart watch. The processor comprises a device to execute certain tasks in the electronic device.

The processor is configured to generate a scoring scale comprising at least three distinct scores concerning disease-related states of the user. The touch-sensitive display is configured to display the scoring scale as generated by the processor enabling a user interaction with the scoring scale by touching the scoring scale at a particular position. The timer is configured for generating timing information for registering timing instances associated with the detected user interaction.

The processor is configured for detecting the user interaction with the touch-sensitive display on or nearby the scoring scale to assign scoring information to the detected user interaction by associating the user interaction with one of the distinct scores.

The processor is further configured to assign first scoring information to a first detected user interaction at a first position of the scoring scale associated with a first score at an first timing instance selected by the user and to maintain this first scoring information during a time interval between the first timing instance and a second timing instance. The second timing instance is also selected by the user and coincides with a second user interaction at a second position of the scoring scale associated with a second distinct score. The processor is configured to assign a second scoring information to the second detected user interaction that is different from the first scoring information.

Another aspect of the invention relates to a method in an electronic user device as described herein, wherein the method comprises the steps of:
generating a scoring scale with at least three distinct scores concerning disease-related states of the user;
displaying the generated scoring scale on a touch-sensitive display of the user device;
detecting a user interaction of a user at or nearby the scoring scale displayed on the touch-sensitive display at an arbitrary time selected by the user;
generating timing information for registering instances of time associated with the detected user interaction at arbitrary times selected by the user;
assigning scoring information to the user interaction by associating the user interaction with one of the distinct scores, wherein first user information is assigned to a first detected user interaction at a first position of the scoring scale associated with a first distinct score at a first timing instance selected by the user and maintaining the first scoring information during a time interval between the first timing instance and a second timing instance;
assigning second scoring information, different from the first scoring information, at the second time instance selected by the user that coincides with a second user interaction at a second position of the scoring scale associated with a second distinct score.

The disclosed electronic user device and method provide a user convenient manner for registering disease-related states of the user. Different from WO 2017/086798, in this manner a diary of the user state as perceived by the user is obtained that is not dependent on measurement data from sensors in the device. Sensor data from the device may be obtained in addition to the diary in order to interpret the diary input data from the user, but the diary as such is solely produced from the user input data.

The user device enables the user to touch the scoring scale once he perceives a change of his or her disease-related state, i.e. the device enables the user to register a change of his or her disease-related state at arbitrary times selected by him. The device maintains scoring information associated with this state as long as the user does not touch a different part of the scoring scale associated with a further distinct score. The user does not need to confirm his or her state as long as the state remains the same, thereby avoiding unnecessary actions by the user and avoiding annoying reminder messages to confirm the state as is done in US 2015/0190084 and WO 2006/088415. The user is only required to operate the device when he perceives a change in his state.

Maintaining the scoring state, in one embodiment, enables the device to present a diary of the user state on the display of the device without an analysis on an external computer system. In this embodiment, the device is enabled to process the scoring information by extrapolating the scoring information associated with one score in time until a point in time for which different scoring information was stored associated with a different score. From that point in time, the different scoring information is extrapolated until a point in time wherein again new scoring information is found.

Maintaining the scoring state also includes, in one embodiment, to store the scoring information together with a time stamp for further processing in the device or in an external computer system.

It should be noted that the at least three distinct scores of the scoring scale provide for an interval scoring scale. The intervals may be of any size, including a (quasi-) continuous scoring scale wherein each position may be a different score. It should be noted that different user interaction with different, nearby, positions may be assigned by either the processor or the external computer system to the same scoring information.

The timing information enables the electronic device or a receiver of the scoring information to generate a diagram wherein the scoring information is displayed along a time scale, i.e. a diary of the user-perceived state can be obtained. The timing information may comprise time stamps that are generated when a user interaction is detected on the scoring scale displayed on the touch-sensitive display. It should be appreciated that any form of timing information that allows the construction of such a diagram may be used. The timer function may be performed by the processor.

In one embodiment of the invention, the processor is further configured to generate at least one of a first graphical element, a second graphical element and a third graphical element for display on the touch sensitive display. Such graphical elements, which may be referred to as icons, may be used by the user to register e.g. medicine intake, food intake or sleep.

The processor is configured to detect a further user interaction with the first, second, and/or third graphical element to generate an event indicator triggered by the user interaction with the first, second or third graphical element.

The timer is configured for generating timing information associated with the user interaction with the first, second and/or third graphical element, such that the event indicator can be correlated in time with the scoring information.

The embodiment enables providing additional information with the scoring information on events that may influence the disease-related state of the user and hence also influence the scoring information. This additional information may e.g. be associated with time instances of medicine intake, food intake and/or sleep by the user. When a doctor studies the scoring information over a time interval along with such events, the additional information may e.g. result in advise of the doctor to change the times of the medicine intake by the user, possibly in relation with the times of the food intake.

In one embodiment, the processor is configured to generate the scoring scale such that the scoring scale is displayed as an arc on the touch-sensitive display. The processor assigns scoring information associated with a good state of the user to a middle part of the arc and assigns scoring information distinct from the good state to parts of the arc on both sides of the middle part of the arc.

For some diseases, such as Parkinson's disease, disease-related states exist on both sides of a good state of the user, viz. an off-state wherein the user experiences rigidity and a dyskinesia state wherein the user experiences superfluous activity. The arc shaped scoring scale provides for a user intuitive scoring scale, particularly because the part of the scoring scale associated with a good state of the user (also referred to as ‘on-state for Parkinson's disease) can be displayed at the top side of the scoring scale, while still having the option to show two lesser scores associated with different disease-related states. In particular, the arc-shaped scoring scale has a first input region provided at the top side of the scoring scale wherein the user device assigns first scoring information to the user selection and a second and third input region at both sides of the first region wherein the user device assigns second and third scoring information to the user selection. The first, second and third scoring information are different, whereas the second and third scoring information relate to non-related states (e.g. stiffness and trembling) of the user. The arc-shaped scoring scale facilitates presentation and selection of these non-related states on a single scale or, in other words, in one dimension.

Moreover, the arc-shaped scoring scale provides for additional length of the scoring scale as compared to a straight line, thereby providing a larger surface area facilitating user input of a particular disease-related state of the scoring scale on a small display, such as the display of a watch. This is particularly important for some diseases, such as Parkinson's disease, wherein the selection ability of the user is limited. Alternatively, the additional length enables the scoring scale to contain more regions associated with different states on the single scale. The scoring scale may be shaped such that it follows the contours of the display of the watch.

In one embodiment, the user device comprises at least one accelerometer configured for registering motion of the user to generate movement information, wherein the timer generates timing information associated with the movement information.

The embodiment enables to provide additional measurements based on movements of the user providing additional information for a doctor along with the scoring information. The accelerometer data may e.g. be used to conduct power measurements to obtain activity information of the user in addition to the scoring information over time.

In one embodiment, the user device contains a network connection unit configured for transmitting at least the scoring information and the associated timing information over a network to an external computer system.

Whereas the scoring information may be stored locally in the electronic device for later display on the display of the user device or for transfer to a computer system, the electronic device is preferably enabled to wirelessly connect to an external, e.g. a remote, computer system. The electronic device may e.g. use a network based on an 802.11x standard (wifi) to upload the scoring information in association with the timing information. The network connection to the external computer system may be continuously active enabling scoring information associated with timing information to be transmitted at all times to the external computer system.

In one embodiment, the user device comprises a watch, wherein the watch optionally comprises a subscriber card, such as a SIM card or an ESIM. The subscriber card allows the watch to connect to a telecommunications network to transmit the scoring information and the associated timing information directly over a telecommunications network to the external computer system.

This embodiment allows the wireless data transfer of the scoring information in association with the timing information wherever coverage of the telecommunications network exists.

In both former embodiments, transmission of new scoring information associated with timing information may e.g. occur when new scoring information is assigned, e.g. after a user interaction with the scoring scale on the touch-sensitive display is detected. Alternatively, the user device may transmit the information at fixed times to the computer system or upon request from the computer system.

In one embodiment, the user device is configured for displaying a medicine intake alert on the touch-sensitive display, wherein the user device, optionally, contains a network connection unit for receiving a medicine intake alert message from an external computer system.

The user device may be programmed to display medicine intake alert messages for the user at programmed times to remind to user to take his medicines. Medicine intake at appropriate times is highly relevant to avoid the user suffering from Parkinson's disease to get into the off-state or the overly active state. The times may be programmed on the user device or via the external computer system. Instead of programming the user device itself, the user may program the external computer system to transmit medicine intake alert messages to the user device at the appropriate times.

In one embodiment, the processor is configured for generating an image of a medicine for the user for display on the touch-sensitive display. The data for generating the image may optionally be received from the external computer system. The images may have been made by the user himself, e.g. with the user device or with another device, and be stored in the user device or been uploaded and stored in the external computer system.

For many diseases, including Parkinson's disease, patients are required to take different medicines, not only in dosage, but also in drugs. This may be confusing for patients and mistakes are regularly made. The embodiment assists user to not only be alerted that medicine should be taken, but also which medicines. It should be appreciated that this embodiment may be executed on its own. That is, a user device is disclosed that shows images of medicines on a display at preprogrammed times. The user device may receive these images from an external computer system and the times at which the user should take the medicines are preprogrammed in the external computer system. The user may have taken the image(s) himself and stored these in the user device and/or uploaded the image(s) to the external computer system. The external computer system may trigger the user device to show the image(s) at the preprogrammed times, e.g. by transmitting a schedule to the user device with times that the image should be generated for display on the user device. To that end, the user device may contain a network connection unit and/or a subscriber module as disclosed above.

In one embodiment, the processor is configured for generating instructions for the user for display on the touch-sensitive display and detect follow-up by the user of the instructions.

The embodiment enables a convenient way for the user to obtain exercise instructions or to conduct tests. The actions of the user may be sensed by one or more sensors in the user device and be transmitted to the external computer system. One example of a test is an instruction for the user to tap the touch-sensitive display quickly in a repeated fashion. The user device may register these actions for use in a rigidity analysis. One example of an exercise is to go cycling and a GPS module in the user device may be used to track the distance.

In one embodiment, the user device contains an alarm module for generating an alarm signal. The user device may contain a dedicated button or graphical element on the touch-sensitive display enabling the user to alert certain predetermined contacts. The alarm module may also be connected to other sensors, e.g. the accelerometer, to alert predetermined contacts when a fall is detected, optionally followed by a period of no motion detection by the accelerometer.

A further aspect of the invention pertains to a wearable electronic user device, such as a watch. The device comprises a processor configured to generate a scoring scale comprising at least three distinct scores concerning disease-related states of the user and a a touch-sensitive display configured to display the scoring scale as generated by the processor to enable a user interaction with the displayed scoring scale. The processor is configured to display the scoring scale in a curved shape and to comprise at least a first input region, a second input region and a third input region associated with the at least three distinct scores enabling the user to select different disease related states of the user. The processor is configured for detecting the user interaction with the first input region, the second input region and the third input region on the touch-sensitive display to assign at least first, second and third scoring information to the detected user interaction by associating the user interaction with one of the distinct scores.

The curved shape of the scoring scale provides for additional length of the scoring scale as compared to a straight line, thereby providing a larger surface area on the touch-sensitive display facilitating user input of a particular disease-related state of the scoring scale on a small display, such as the display of a watch. This is particularly important for some diseases, such as Parkinson's disease, wherein the selection ability of the user is limited. Alternatively, the additional length enables the scoring scale to contain more regions associated with different states on the single scale.

In one embodiment, the scoring scale is shaped to substantially align with a boundary of the touch-sensitive display. In one example, the user device comprises a watch with an oval or circular display and the scoring scale is curved along at least a part of the boundary of the touch-sensitive display to make optimal use of the available area of the display. For example, in one embodiment, the scoring scale is arc shaped.

One further aspect of the invention pertains to an external computer system for use in communication with a user device.

The external computer system comprises a receiver for receiving at least the scoring information assigned by the processor of the user device associated with the timing information. The external computer system further comprises means for generating a diagram showing the variation of the assigned scoring information over a time interval based on the timing information.

In one embodiment, the receiver of the computer system is configured for receiving at least one event indicator associated with timing information as generated by the processor of the user device. The means for generating the diagram are configured for placement of the event indicator in the diagram with the scoring information in association with the timing information.

The external computer system enables a doctor to have access to the scoring information over considerable periods of time for several users of the user devices disclosed herein. This information, combined where possible with event detections (medicine intake, food intake, sleep) and measurements (from e.g. the accelerometer) allows a doctor to advise on changes in behavior of the patient when the experiences problems with his or her disease.

In one embodiment, the external computer system is configured for generating average scoring information over a time interval based on scoring information assigned by the processor over multiple identical time intervals received by the receiver of the computer system from the user device.

The average scoring information facilitates the doctor to recognize patterns in the user behavior and allows a better advice for changes in said behavior.

In one embodiment, the external computer system is configured for transmitting a medicine intake alert message to and/or data for generating an image of the medicine at the user device. It should be noted that the alert message may be part of a scheme for generating messages that is transmitted to the user device indicating times at which the alert message should be generated in the user device. In one embodiment, the point in time for transmitting the medicine intake alert message and/or the data for generating the image is determined based on the scoring information and/or one or more of the event indicators that were received from the user device and/or the average scoring information determined at the external computer system. The latter embodiment enables automatic changes in the timing of the medicine alert messages based upon an analysis of the scoring information over time and the event indicators.

In one embodiment, the external computer system is configured for generating an interface comprising data from users of a plurality of user devices, wherein the users and/or user devices are distinguished in the interface based on the score information assigned by the processors of the respective user devices.

The embodiment provides a dynamic user interface for a doctor and allows a doctor to immediately recognize the users suffering from a particular state at a particular moment in time. For example, a doctor can immediately observe all user having tapped the “off-state” at the scoring scale on their devices when he logs on onto the computer system. As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

### BRIEF DESCRIPTION OF THE DRAWINGS

In the drawings:

FIG. 1 depicts an electronic user device for registering disease-related states of a user and an external computer system according to an embodiment of the invention;

FIGS. 2A and 2B resp. show a flow chart of a method to obtain scoring information and the obtained scoring information;

FIG. 2C is a schematic illustration of a user interface of a computer receiving scoring information diagrams from an external computer system;

FIGS. 3A-3C depict a watch configured for registering disease-related states according to an embodiment of the invention.

FIG. 4 is a picture of diagrams with scoring information over several time intervals and a diagram with average scoring information.

FIG. 5 is a picture of diagrams with scoring information over several time intervals combined with accelerometer measurements; and

FIG. 6 depicts a block diagram illustrating an exemplary data processing system that may be used with embodiments described in this disclosure.

### DETAILED DESCRIPTION

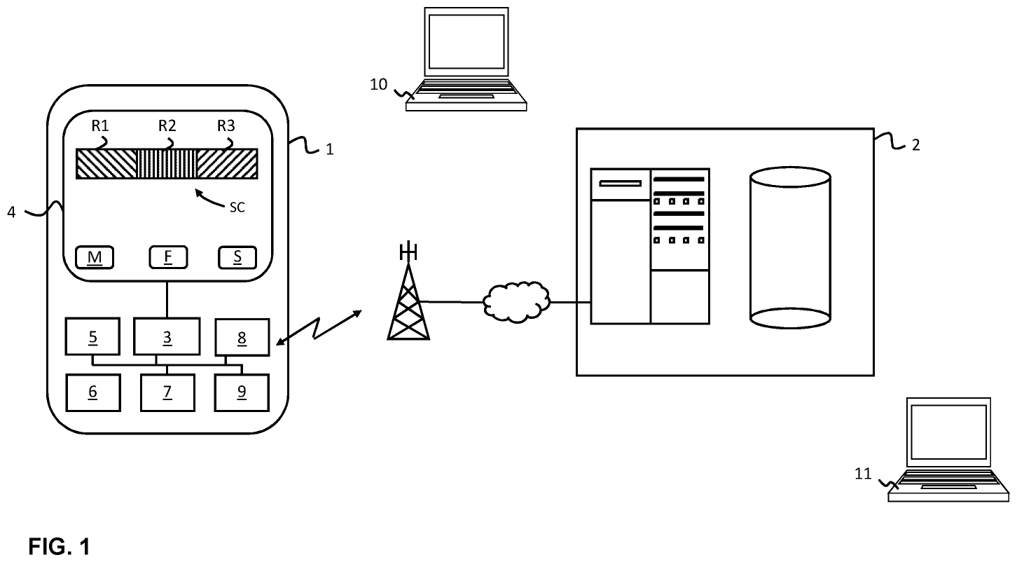

FIG. 1 is a schematic illustration of an electronic user device 1 for registering disease-related states of a user and an external computer system 2 according to an embodiment of the invention. The user device 1 is preferably a wearable electronic user device comprising a processor 3, a touch-sensitive display 4 and a timer 5. The wearable electronic user device 1 may e.g. be a smart phone or a smart watch. Since the user device 1 is a wearable device, a battery 6 is included. Wearable user device 1 also includes memory 7 for storing computer code to enable user device 1 to execute the functions disclosed herein as well as for storing or buffering data generated and assigned for later transmission by the user device 1. For external communications, electronic user device 1 comprises a network connection unit 8 in order to facilitate communications over a network with the external, e.g. remote, computer system 2. Network connection unit 8 may include a subscriber identity module, e.g. a SIM card or ESIM, for communication over a telecommunications network. The electronic user device 1 also contains a sensor 9, which may represent a plurality of sensors, such as an accelerometer and/or a GPS module.

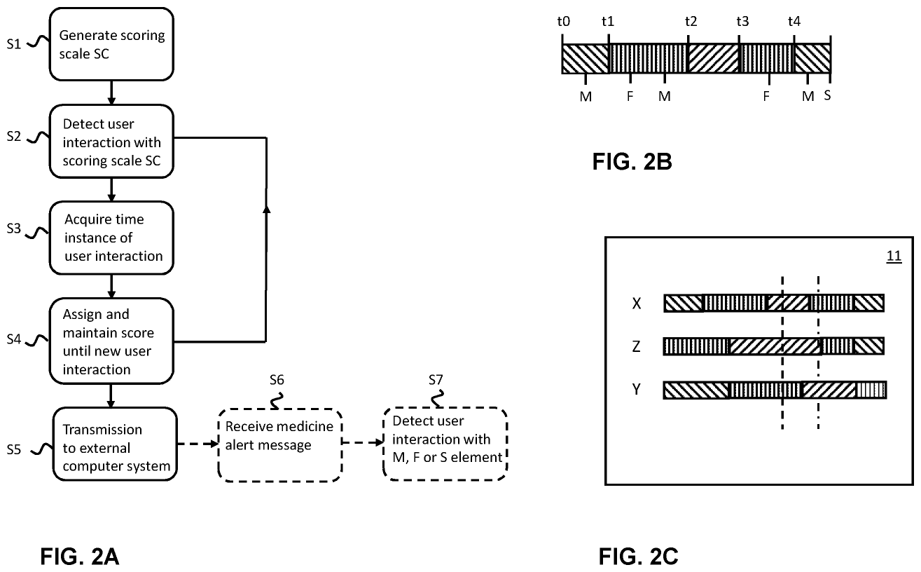

Touch-sensitive display 4 displays a scoring scale SC on the display to enable user interaction therewith. The scoring scale SC comprises three distinct regions R1, R2 and R3 that a user may tap to register his or her disease-related state as will be explained in more detail below with reference to FIGS. 2A and 2B. The distinct regions can be separated by color, using textual indications or in other ways.

The scoring scale SC may also be a continuous scoring scale wherein (almost) every single position on the scoring scale relates to a different score, e.g. a different scoring value or variable. The processor may assign scoring information to this scoring value, wherein distinct scoring values may ultimately be assigned to the same scoring information. In other words, different registered variables can be assigned to a single interval variable. For example, the scoring scale SC of FIG. 1 would be a continuous scoring scale, scoring values from −1 (representing the extreme left part of the scoring scale) via 0 (the middle of the scoring scale) to +1 (the extreme right of the scoring scale) may be obtained by the processor 3. If a user would tap a position corresponding to scoring value −0.1 or +0.1, the processor 3 may generate scoring information corresponding to a good score to both interactions.

Touch-sensitive display 4 also displays a plurality of graphical elements M, F and S that a user may tap to register medicine intake, food intake and sleep respectively.

External computer system 2 comprises a server and storage for storing information received from user devices, such as user device 1. Computer system 2 provides access to this information for the user via user device 1 or another device 10 and also provides access for third parties using device 11. Examples of such third parties are doctors or other care takers. External computer system 2 may also transmit messages or information to the user device 1, such as medicine alert messages, possible including images of medicines.

Referring now to FIG. 2A, step S1 involves the processor 3 of user device 1 to generate scoring scale SC on the touch-sensitive display 4 with three distinct scores, shown by regions R1, R2 and R3, concerning distinct disease related states of the user.

In step S2, the processor 3 detects a user interaction with the scoring scale SC displayed on the touch-sensitive display 4. In one example of a user suffering from Parkinson's disease, with a tap on region R1, the user registers that the user is in an off-state, with a tap on region R2 that the user is good and with a tap on region R3 that the user is overly active.

In step S3, timing information is generated by acquiring instances of time when the user interacts with the scoring scale SC. Such time instances are shown as t0, t1, t2, t3 and t4 in FIG. 2B.

In step S4, the processor 3 assigns scoring information to the user interaction by associating the user interaction with one of the distinct scores, represented by regions R1, R2 and R3. The scoring information may have any form and format, e.g. numeric values, (color) codes, etc. The processor 3 maintains the scoring information over a time interval until a second user interaction with the scoring scale SC is detected. When the user interaction is with a different part of the scoring scale SC, i.e. a different region R1, R2, R3 than previously detected, the processor assigns second scoring information to the tap which is different from the previous scoring information and then maintains this second user information until a subsequent user interaction is detected as shown by the loop from step S4 back to step S2.

In step S5, the scoring information and the associated timing information are transmitted to external computer system 2.

Optionally, as shown in step S6, the user device 1 may receive a medicine alert message from external computer system 2 via network connection unit 8. The medicine alert message may include an image of the particular medicine that the user should take at this point in time. Other information may also be received from computer system 2, e.g. instructions for the user for display on the touch-sensitive display 4. The external computer system may detect follow-up by the user of the instructions. One example of a test is an instruction for the user to tap the touch-sensitive display 4 quickly in a repeated fashion. The user device 1 may register these actions for use in a rigidity analysis at the computer system 2.

Optionally, as shown in step S7, the processor detects another user interaction with one or more of the graphical elements M, F or S. It is assumed that timer 5 generates timing information associated with this user interaction such that indications of medicine intake, food intake and or sleep can be presented on the same time axis as the scoring information assigned in step S4. Like the scoring information, the event indicators may be transmitted to external system 2 (not shown in FIG. 2A). If step S6 was performed, the external computer system 2 may verify whether the medicine alert message was followed up by the user by checking whether the event indicator corresponding to graphical element M is received. If too much time exists between the transmission of the medicine alert message and the receipt of the event indictor representing medicine intake M, external computer system 2 may generate an alarm signal to a care taker. Furthermore, the external computer system 2 may adjust the timing of the transmission of the medicine alert message if follow up by the user is too slow.

A diagram with the scoring information may be presented on the display of user device 1 or may be generated by external computer system 2 for display on computers 10 and/or 11. An exemplary diagram is shown in FIG. 2B. The total time interval between t0 and the end of the diagram, indicated by S representing a sleep event generated by the user interacting with graphical element S, is assumed to represent one day.

During a first time interval between times t0, e.g. when the user wakes up, and time t1, the user is in an off-state, i.e. the user feels stiff and rigid, the user taps region R1 on time t0 on the scoring scale SC displayed on touch-sensitive display 4 in step S1. Processor 3 assigns the corresponding scoring information (step S4) and transmits the scoring information to the external computer system 2 including the timing information (step S5). At some point in time during this time interval, the user takes his medicine (e.g. levodopa) and taps graphical element M, which event information is also included in the diagram. During this time interval, processor 3 maintains the scoring information corresponding to R1 without requesting the user to confirm his state. If the user would again tap the region R1 on the scoring scale SC, processor 3 may maintain or reassign the same scoring information and, either transmit this scoring information again to external computer system 2 or not. User device 1 or external computer system 2 may inform the user that registration of his or her state was already done.

At time instance t1, the user starts to feel good (possible as a result of taking the medicine) and taps corresponding region R2 on scoring scale SC on touch-sensitive display 4 of the user device 1. Processor 3 no longer maintains the scoring information of time interval t0-t1, but assigns new scoring information corresponding to the good state represented by region R2 on the scoring scale, along with time information generated by the timer 5 and transmits this information to external computer system 2. Processor 3 maintains the new scoring state until the user taps a different region again, i.e. region R1 or R3, as occurs at time instance t2 in the diagram of FIG. 2B. During the time interval t1-t2, the user also registers food intake F by tapping graphic element F on touch-sensitive display 4. This event is also transmitted to external computer system 2 along with time information such that also the food intake can be correlated in time with the scoring information. During this time interval t1-t2, the user also takes another dose of levodopa because he or she desires to continue the good state and registers taking this medicine again by tapping graphical element M.

Quite soon after administering the medicine, most likely because too short a time interval existed between the two levodopa intakes by the user, the user starts to feel overly active at time instance t2 and registers his new state by tapping region R3 on scoring scale SC displayed on the touch-sensitive display 4 of the user device 1. Processor 3 maintains the scoring information until time t3 when the user taps region R2 on the scoring scale SC because the overly active state ceases (the too high levodopa dose has decreased to normal level). Another food intake is registered during the time interval t3-t4.

The user has now been cautious not to take another medicine dose but waits too long such that the rigidity and stiffness return at time instance t4 registered by the user by tapping region R1 again. The user takes another medicine before he or she goes to sleep and registers both events, i.e. medicine intake M and sleep S.

FIG. 2C is a schematic illustration of a user interface generated by external computer system 2 and presented to computer display of computer 11 of a doctor being responsible for patients X, Y and Z each employing a user device 1. After the doctor logs on to the external computer system 2, the doctor may display the diagrams of users X, Y and Z on the display of computer 11. The diagram of user X corresponds to the diagram of FIG. 2B.

The doctor may arrange the order in which the users are presented in the user interface according to the present states as shown in FIG. 2C. At a point in time in time interval t2-t3, indicated by the dashed line, in the diagram of user X, user X and also user Z is in an overly active state, whereas user Y reports a good state. Hence, the diagrams of users X and Z are presented before the diagram of user Y. At a later moment in time, e.g. in time interval t3-t4 of user X, indicated by the dashed-dotted line, the diagrams of users Z and Y are presented before the diagram of user X, because both users Z and Y report being overly active.

Another method for the doctor to arrange the order is to select patients on the basis of the change in percentage of time intervals wherein the patients register good states. In this way, the doctor get a good overview of patients that need attention first.

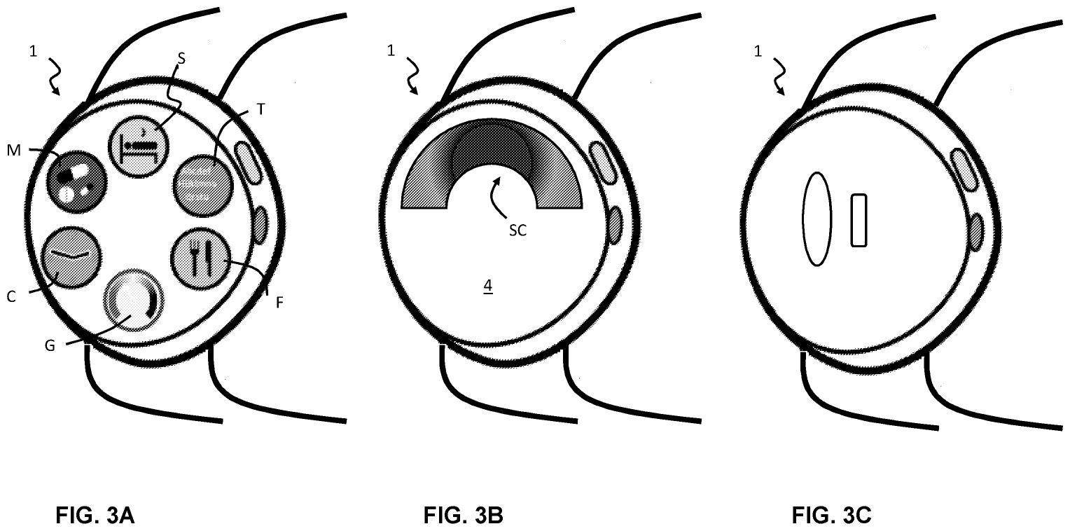

FIG. 3A is a schematic illustration of electronic user device 1 as a smart watch embodiment. FIG. 3A shows the touch-sensitive display 4 generated by processor 3 displaying a default watch interface with graphical elements M, F and S as described with reference to FIGS. 1, 2A and 2B and in addition a graphical element C enabling the user to generate a normal clock interface, a graphical element T enabling the user to transmit free or standardized text to the remote computer system to add comments to the scoring information as well as a graphical element G to trigger processor 3 to generating the scoring scale SC for the touch-sensitive display 4.

When the user taps graphical element G on the smart watch 1, processor 3 generates the scoring scale SC as shown in FIG. 3B that is displayed on touch-sensitive display 4. Scoring scale SC in FIG. 3B has a continuous scoring scale SC instead of the 3-state scoring scale of the user device 1 of FIG. 1. The continuity may ultimately be on the level of the pixel density of the touch-sensitive display 4.

The processor 3 generates the scoring scale SC in FIG. 3B such that the scoring scale is displayed as an arc on the touch-sensitive display 4. The scoring scale SC is substantially aligned with the boundary of the touch-sensitive display 4. The processor 3 assigns scoring information associated with a good state of the user to a middle part of the arc and assigns scoring information distinct from the good state to parts of the arc on both sides of the middle part of the arc. As mentioned above, for Parkinson's disease, disease-related states exist on both sides of a good state of the user, viz. an off-state wherein the user experiences rigidity and a dyskinesia state wherein the user experiences superfluous activity. Other disease-related states (e.g. seriousness of headache, dizziness, etc.) for other diseases have also been considered for registration on sides of the good state.

The arc shaped scoring scale SC of FIG. 3B provides for a user intuitive scoring scale, particularly because the part of the scoring scale associated with a good state of the user (also referred to as ‘on-state’ for Parkinson's disease) can be displayed at the top side of the scoring scale, while still having the option to show two lesser scores associated with different disease-related states. Touch-sensitive display 4 may be a color display and the different states may be indicated by colors, e.g. colors in the yellow spectrum for the off-state flowing over to colors in the green spectrum for the good state flowing over to colors in the red spectrum for the overly active state.

Moreover, the arc-shaped scoring scale SC provides for additional length of the scoring scale SC as compared to a straight line, thereby providing a larger surface area facilitating user input of a particular disease-related state of the scoring scale on a small display, such as the display of a watch. This is particularly important for some diseases, such as Parkinson's disease, wherein the selection ability of the user is limited. Alternatively, the additional length enables the scoring scale to contain more regions, e.g. five or seven regions, associated with different states on the single scale. In one example, the user can select two or three degrees of stiffness and two or three degrees of activeness on the scoring scale, while the area for selection of these scores is still reasonably large.

FIG. 3C shows a particular example, wherein the processor 3 of user device 1 generates an image of medicines that the user should take shortly. The user device 1 may be programmed to display medicine intake alert messages for the user at programmed times to remind to user to take his medicines. Medicine intake at appropriate times is highly relevant to avoid the user suffering from e.g. Parkinson's disease to get into the off-state or the overly active state. The times may be programmed on the user device 1 or via the external computer system 2. Instead of programming the user device 1 itself, the user may program the external computer system 2 to transmit medicine intake alert messages to the user device 1 at the appropriate times using the computer 10 connected to the external computer system 2.

The processor 3 is configured for generating an image of a medicine for the user for display on the touch-sensitive display. The data for generating the image may optionally be received from the external computer system 2.

For many diseases, including Parkinson's disease, patients are required to take different medicines, not only in dosage, but also in drugs. This may be confusing for patients and mistakes are regularly made. The embodiment of FIG. 3C assists a user to not only be alerted that medicine should be taken, but also shows which medicines he or she should take.

As mentioned above, the external computer system 2 may be configured for transmitting a medicine intake alert message to and/or data for generating an image of the medicine at the user device 1. In one embodiment, the point in time for transmitting the medicine intake alert message and/or the data for generating the image is determined based on the scoring information and/or one or more of the event indicators M, F, S that were received from the user device and/or the average scoring information determined at the external computer system. The latter embodiment enables automatic changes in the timing of the medicine alert messages based upon an analysis of the scoring information over time and the event indicators.

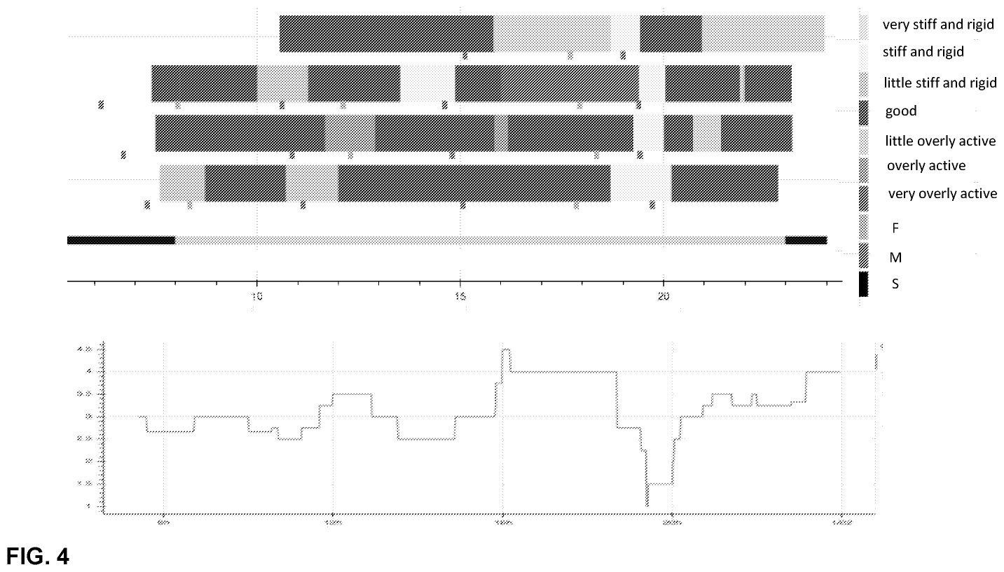

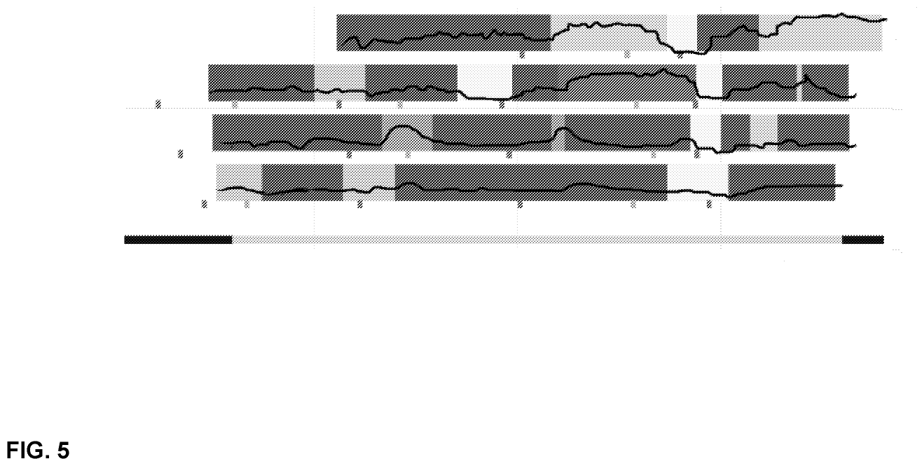

FIG. 4 illustrates diagrams with scoring information spanning time intervals of four consecutive days for a particular user using the user device of FIGS. 3A and 3B wherein the processor assigns seven levels of scoring information to registrations on the continuous scoring scale SC. For example, scoring values on the left of zero are assigned to three different scoring information (e.g. −1 to −0.75=very stiff and rigid; −0.75 to −0.5=stiff and rigid; −0.5 to −0.2—little stiff and rigid), such that the user can register a degree of stiffness/rigidity and a degree of activeness using the scoring scale SC as shown in FIG. 3B. The same is done for the right-hand side of the scoring scale for registering degrees of overly activeness. It should be noted that the grey scale variation shown in the scoring scale SC of FIG. 3B on the user device 1 does not entirely correspond to the gray scales shown in FIGS. 4 and 5. The lower bar in FIG. 4 indicates night (black) and day (grey).

From the different diagrams in FIG. 4, it is clear that the user registration of the disease-related state differs considerably between the days. For the doctor to recognize a pattern, the user device 1 or external computer system 2 may calculate an average scoring information during a time interval (e.g. during a day) by assigning values to each of the registered states and computing the average for several points in time during the time interval. For example, numerical values 0-2 may be attributed to the stiff/rigidity regions, 3 to the good region and 4-6 to the overly active regions such that an average can be computer for each moment during the time interval over the registered four days. The result is shown at the bottom diagram of FIG. 4. From the average, the doctor may observe that the user is generally overly active around 16:00 h, indicating that the medication dose and/or intake time should be adjusted. Furthermore, the doctor would note the occurrence of an off-state in the early evening hours and may decide on further action as well.

FIG. 5 shows similar diagrams, now also including measurement of accelerometer 9 of user device 1 on the same time scale as the scoring information and the event indications. The accelerometer measurements can be used to confirm the states registered by the user when tapping the scoring scale SC on touch-sensitive display 4. When the user reports an overly active state, the trend in the accelerometer measurement would show more movement, whereas less movement would be expected when the user registers an off-state.

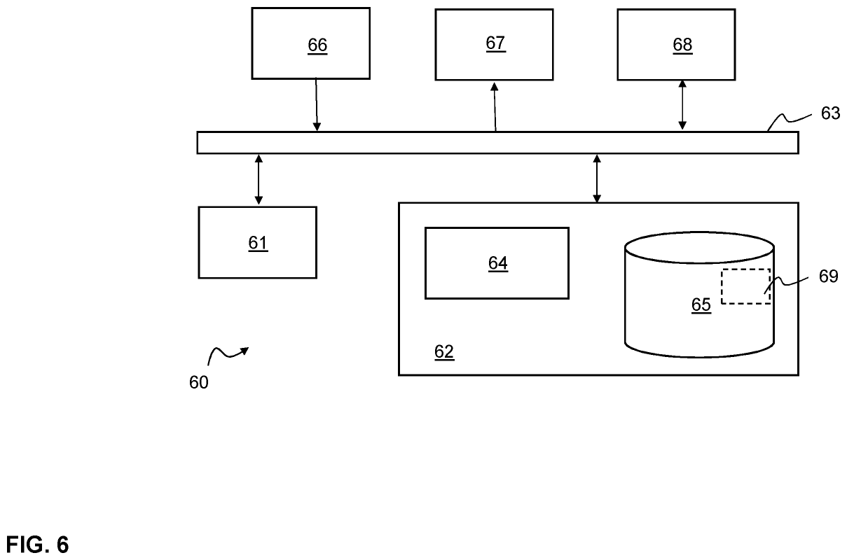

FIG. 6 is a block diagram illustrating an exemplary data processing system that may be used as described in this disclosure. Data processing system 60 may include at least one processor 61 coupled to memory elements 62 through a system bus 63. As such, the data processing system may store program code within memory elements 62. Further, processor 61 may execute the program code accessed from memory elements 62 via system bus 63. In one aspect, data processing system may be implemented as a computer that is suitable for storing and/or executing program code. It should be appreciated, however, that data processing system 60 may be implemented in the form of any system including a processor and memory that is capable of performing the functions described within this specification.

One example of data processing system may be user device 1 and/or external computer system 2.

Memory elements 62 may include one or more physical memory devices such as, for example, local memory 64 and one or more bulk storage devices 65. Local memory may refer to random access memory or other non-persistent memory device(s) generally used during actual execution of the program code. A bulk storage device may be implemented as a hard drive or other persistent data storage device. The processing system 60 may also include one or more cache memories (not shown) that provide temporary storage of at least some program code in order to reduce the number of times program code must be retrieved from bulk storage device 65 during execution.

Input/output (I/O) devices depicted as input device 66 and output device 67 optionally can be coupled to the data processing system. Examples of input device may include, but are not limited to, for example, a keyboard, a pointing device such as a mouse, or the like. Examples of output device may include, but are not limited to, for example, a monitor or display, speakers, or the like. Input device and/or output device may be coupled to data processing system either directly or through intervening I/O controllers. A network adapter 68 may also be coupled to data processing system to enable it to become coupled to other systems, computer systems, remote network devices, and/or remote storage devices through intervening private or public networks. The network adapter may comprise a data receiver for receiving data that is transmitted by said systems, devices and/or networks to said data and a data transmitter for transmitting data to said systems, devices and/or networks. Modems, cable modems, and Ethernet cards are examples of different types of network adapter that may be used with data processing system 60.

As pictured in FIG. 6, memory elements 62 may store an application 69. It should be appreciated that data processing system 60 may further execute an operating system (not shown) that can facilitate execution of the application. Application 69, being implemented in the form of executable program code, can be executed by data processing system 60, e.g., by processor 62. Responsive to executing application, data processing system may be configured to perform one or more operations to be described herein in further detail.

In one aspect, for example, data processing system 60 may represent a client data processing system. In that case, application 69 may represent a scoring information assignment application that, when executed, configures data processing system 60 to perform the various functions described herein with reference to a “client”, i.e. user device 1. Examples of a client can include, but are not limited to, a personal computer, a portable computer, a mobile phone, a smart watch, smart glasses, VR glasses, or the like.

In another aspect, data processing system may represent a server. For example, data processing system may represent an (HTTP) server in which case application 69, when executed, may configure data processing system to perform (HTTP) server operations, e.g. in external computer system 2. In another aspect, data processing system may represent a module, unit or function as referred to in this specification.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A wearable electronic user device comprising:
a processor configured to generate a scoring scale comprising at least three distinct scores concerning disease-related states of a user;
a touch-sensitive display operably coupled to the processor and configured to display the scoring scale as generated by the processor to enable a user interaction with the displayed scoring scale;
a timer operably coupled to the processor and configured for generating timing information for registering timing instances associated with the detected user interaction;
wherein the processor is configured for detecting the user interaction with the touch-sensitive display on or nearby the scoring scale to assign scoring information to the detected user interaction by associating the user interaction with one of the distinct scores;
wherein the processor is further configured to assign first scoring information to a first detected user interaction at a first position of the scoring scale associated with a first score at a first timing instance and to maintain this first scoring information during a time interval between the first timing instance and a second timing instance, wherein the second timing instance coincides with a second user interaction at a second position of the scoring scale associated with a second distinct score, and
wherein the processor is configured to assign a second scoring information to the second detected user interaction that is different from the first scoring information.

2. The device according to claim 1, wherein the processor is further configured to generate at least one of:
a first graphical element for display on the touch sensitive display;
a second graphical element for display on the touch sensitive display;
a third graphical element for display on the touch sensitive display;
wherein the processor is configured to detect a further user interaction with the first, second, and/or third graphical element to generate an event indicator triggered by the user interaction with the first, second or third graphical element;
wherein the timer is configured for generating timing information associated with the user interaction with the first, second and/or third graphical element, such that the event indicator can be correlated in time with the scoring information.

3. The device according to claim 1, wherein the processor is configured to generate the scoring scale such that the scoring scale is displayed as an arc on the touch-sensitive display, wherein the processor assigns scoring information associated with a good state of the user to a middle part of the arc and assigns scoring information distinct from the good state to parts of the arc on both sides of the middle part of the arc.

4. The device according claim 1, wherein the device comprises at least one accelerometer operably coupled to the processor and configured for registering movements of the user and to generate movement information, wherein the timer generates timing information associated with the movement information.

5. The device according to one claim 1, wherein the device comprises contains a network connection unit operably coupled to the processor and configured for transmitting at least the scoring information and the associated timing information over a network to an external computer system.

6. The device according to claim 5, wherein the devices comprises a watch, wherein the watch optionally comprises a subscriber card to transmit the scoring information and associated timing information to the external computer system directly over a telecommunications network.

7. The device according to claim 1, wherein the device is further configured for displaying a medicine intake alert on the touch-sensitive display.

8. The device according to claim 7, wherein the processor is further configured for generating an image of a medicine for the user for display on the touch-sensitive display.

9. The device according to claim 1, wherein the processor is further configured for generating instructions for the user for display on the touch-sensitive display and detect follow-up by the user of the instructions.

10. The device according to claim 1, wherein the device comprises an alarm module for generating an alarm signal.

11. A wearable electronic user device comprising:
a processor configured to generate a scoring scale comprising at least three distinct scores concerning disease-related states of the user;
a touch-sensitive display operably coupled to the processor and configured to display the scoring scale as generated by the processor to enable a user interaction with the displayed scoring scale;
wherein the scoring scale is displayed as a curved shape and comprises at least a first input region, a second input region and a third input region associated with the at least three distinct scores enabling the user to select different disease related states of the user; and
wherein the processor is configured for detecting the user interaction with the first input region, the second input region and the third input region on the touch-sensitive display to assign at least first, second and third scoring information to the detected user interaction by associating the user interaction with one of the distinct scores.

12. The wearable electronic user device according to claim 11, wherein the scoring scale is substantially aligned with a boundary of the touch-sensitive display.

13. The wearable electronic user device according to claim 11, wherein the scoring scale is an arc-shaped scoring scale.

14. (canceled)

15. A system comprising:
a wearable electronic user device comprising:
a processor configured to generate a scoring scale comprising at least three distinct scores concerning disease-related states of a user;
a touch-sensitive display operably coupled to the processor and configured to display the scoring scale as generated by the processor to enable a user interaction with the displayed scoring scale;
a timer operably coupled to the processor and configured for generating timing information for registering timing instances associated with the detected user interaction;
wherein the processor is configured for detecting the user interaction with the touch-sensitive display on or nearby the scoring scale to assign scoring information to the detected user interaction by associating the user interaction with one of the distinct scores;
wherein the processor is further configured to assign first scoring information to a first detected user interaction at a first position of the scoring scale associated with a first score at a first timing instance and to maintain this first scoring information during a time interval between the first timing instance and a second timing instance, wherein the second timing instance coincides with a second user interaction at a second position of the scoring scale associated with a second distinct score, and
wherein the processor is configured to assign a second scoring information to the second detected user interaction that is different from the first scoring information; and
an external computer system for use in communication with the user device, wherein the external computer system comprises:
a receiver configured to receive at least the scoring information assigned by the processor of the user device associated with the timing information; and
means for generating a diagram showing a variation of the assigned scoring information over a time interval based on the timing information.

16. The system according to claim 15, wherein the receiver is further configured for receiving at least one event indicator associated with timing information as generated by the processor of the user device and wherein the means for generating the diagram are configured for placement of the event indicator in the diagram with the scoring information in association with the timing information.

17. The system according to claim 15, wherein the external computer system is configured for generating average scoring information over a time interval based on scoring information assigned by the processor over multiple identical time intervals received by the receiver of the computer system from the user device.

18. The system according to claim 15, wherein the external computer system is configured for transmitting a medicine intake alert message to and/or data for generating an image of the medicine at the user device.

19. The system according to claim 18, wherein a point in time for transmitting the medicine intake alert message and/or the data for generating the image is determined based on the scoring information and/or one or more of the event indicators that were received from the user device and/or the average scoring information determined at the external computer system.

20. The system according to claim 15, wherein the external computer system is configured for generating an interface comprising data from users of a plurality of user devices, wherein the users and/or user devices are distinguished in the interface based on the score information assigned by the processors of the respective user devices.

21. A method in an electronic user device comprising:
generating a scoring scale with at least three distinct scores concerning disease-related states of a user;
displaying the generated scoring scale on a touch-sensitive display of the user device;
detecting a user interaction of a user at or nearby the scoring scale displayed on the touch-sensitive display;
generating timing information for registering instances of time associated with the detected user interaction;
assigning scoring information to the user interaction by associating the user interaction with one of the distinct scores, wherein first scoring information is assigned to a first detected user interaction at a first position of the scoring scale associated with a first distinct score at a first timing instance and maintaining the first scoring information during a time interval between the first timing instance and a second timing instance, wherein the second timing instance coincides with a second user interaction at a second position on the scoring scale associated with a second distinct score; and
assigning second scoring information to the second user interaction, different from the first scoring information, at the second timing instance.

22. (canceled)
